(19) United States
(12) Patent Application Publication
(10) Pub. No.: US 2014/0187749 A1
(43) Pub. Date: Jul. 3, 2014

(54) SINGLE UNIT CHROMATOGRAPHY ANTIBODY PURIFICATION

(71) Applicants: Maria Perlasca Islas, Echt (NL); Diderik Reinder Kremer, Echt (NL); Mark K. Doeven, Echt (NL); Henderik E. Veenstra, Echt (NL)

(72) Inventors: Maria Perlasca Islas, Echt (NL); Diderik Reinder Kremer, Echt (NL); Mark K. Doeven, Echt (NL); Henderik E. Veenstra, Echt (NL)

(73) Assignee: DSM IP ASSETS B.V., Heerlen (NL)

(21) Appl. No.: 14/126,677

(22) PCT Filed: Jun. 8, 2012
(86) PCT No.: PCT/EP2012/060885

§ 371 Date: Mar. 21, 2014

(30) Foreign Application Priority Data
Jun. 16, 2011 (EP) 11170239.5

(51) Int. Cl. C07K 1/36

(52) U.S. Cl. CPC C07K 1/36
USPC 530/387.1

(57) ABSTRACT

The present invention relates to a method for the purification of antibodies from a protein mixture produced in a bioreactor, at least comprising the steps of intermediate purification and polishing, wherein the intermediate and polishing step comprises in-line anion exchange chromatography (AEX) treatment and mixed mode chromatography (MiMo) treatment in flow through mode. The present invention further relates to a single operational unit comprising both an anion exchange chromatography part and a mixed mode chromatography part, which are serially connected, wherein the unit comprises an inlet at the upstream end of the anion exchange chromatography part and an outlet at the downstream end of the mixed mode chromatography part and wherein the unit also comprises an inlet between the anion exchange chromatography part and the mixed mode chromatography part.

The present invention relates to a method for single unit purification of antibodies and to equipment which can be used in this method.

The purification of monoclonal antibodies, produced by cell culture, for use in pharmaceutical applications is a process involving a large number of steps. The antibodies are essentially to be freed from all potentially harmful contaminants such as proteins and DNA originating from the cells producing the antibodies, medium components such as insulin, PEG ethers and antifoam as well as any potentially present infectious agents such as viruses and prions.

Typical processes for purification of antibodies from a culture of cells producing these proteins are described in BioPharm International June 1, 2005, “Downstream Processing of Monoclonal Antibodies: from High Dilution to High Purity.”

As antibodies are produced by cells, such as hybridoma cells or transformed host cells (like Chinese Hamster Ovary (CHO) cells, mouse myeloma-derived NSO cells, Baby Hamster Kidney cells and human retina-derived PER.C6® cells), the particulate cell material will have to be removed from the cell broth, preferably early in the purification process. This part of the process is indicated here as “clarification”. Subsequently or as part of the clarification step the antibodies are purified roughly to at least about 80%, usually with a binding plus eluting chromatography step (in the case of IgG often using immobilized Protein A). This step, indicated here as “capturing” not only results in a first considerable purification of the antibody, but may also result in a considerable reduction of the volume, hence concentration of the product. Alternative methods for capturing are for example Expanded Bed Adsorption (EBA), 2-phase liquid separation (using e.g. polyethyleneglycol) or fractionated precipitation with lyotropic salt (such as ammonium sulfate).

Subsequent to clarification and capturing, the antibodies are further purified. Generally, at least 2 chromatographic steps are required after capturing to sufficiently remove the residual impurities. The chromatographic step following capturing is often called intermediate purification step and the final chromatographic step generally is called the polishing step. Each of these steps is generally performed as single unit operation in batch mode and at least one of these steps generally is carried out in the binding plus eluting mode. In addition, each chromatographic step requires specific loading conditions with respect to e.g. pH, conductivity etc. Therefore, extra handling has to be performed prior to each chromatography step in order to adjust the load to the required conditions. All of this mentioned makes the process elaborate and time consuming. The impurities generally substantially removed during these steps are process derived contaminants, such as host cell proteins, host cell nucleic acids, culture medium components (if present), protein A (if present), endotoxin (if present), and micro-organisms (if present). Several methods for such purification of antibodies have been described in recent patent publications.

WO 2010/062244 relates to an aqueous two phase extraction augmented precipitation process for isolation and purification of proteins like monoclonal antibodies. For subsequent further purification of antibodies two alternatives are described: (1) cation exchange chromatography in bind and elute mode, followed by anion exchange in flow through mode, or (2) first multimodal (or mixed-mode) chromatography in flow through mode, followed by anion exchange in flow-through mode. The two chromatographic units of alternative (2) do not operate as one single unit operation and none is used for polishing purposes.

WO 2005/044856 relates to the removal of high-molecular weight aggregates from an antibody preparation, using a hydroxyapatite resin optionally in combination with anion exchange chromatography. Both chromatography treatments were described amongst others as flow-through processes, however they were described to be carried out as separate operations.

WO 2011/017514 relates to the purification of antibodies and other Fc-containing proteins by subsequent in-line cation and anion exchange chromatography steps. Both chromatography treatments were generally carried out as bind-and-elute separations, although the second step may be operated as a flow-through process.

WO2005/082483 relates to the purification of antibodies by two subsequent mixed mode chromatography steps, wherein the chromatography material of the first step is a mixed-mode cation exchange resin having both cation-exchanging groups and aromatic groups by which binds the antibodies can be bound and the chromatography material of the second step is a mixed mode anion exchange resin. The second chromatography step can be carried out in flow-through mode. The two chromatography steps are described as separate operations.

Disadvantages of the methods described above are long operation time, high variable costs and high fixed cost (due to labor costs).

According to one embodiment of the present invention, very efficient removal of residual impurities from cell culture-produced antibodies can be achieved by using serial, in-line anion exchange chromatography (AEX) and mixed-mode (MiMo) chromatography both in the flow-through mode. In-line conditioning of the flow-through from the AEX step (e.g. by mixing of a suitable buffer) prior to the MiMo chromatographic step is used to adjust the flow-through to the right conditions with respect to pH and conductivity for the MiMo chromatography.

Advantages of this novel method are considerable reduction of the operation time and labor and hence lower operational costs. In addition, smaller (and thus less costly) chromatographic units are required, since all units operate in flow-through mode which requires only sufficient binding capacity for the impurities and not for the product.

Therefore, the present invention can be defined as a method for the purification of antibodies from a cell broth produced in a bioreactor, at least comprising the steps of intermediate purification and polishing, wherein the novel purification step comprises combined serial in-line AEX and MiMo chromatography. This can be carried out by applying an AEX chromatography step yielding as a flow-through fraction a separation mixture, serial in-line followed by a MiMo chromatography step yielding as a flow-through fraction a purified antibody preparation and wherein the purified antibody preparation is subjected to at least one further purification step.

Hence, in the context of the present invention, the “separation mixture” is the solution resulting from the first chromatography step according to the invention, and the “purified antibody preparation” is the solution resulting from the second chromatography step according to the invention. It is intended to adhere to this terminology throughout the present application.

Prior to the first chromatography step, the cell broth produced in the bioreactor generally will be clarified (i.e. freed from all cellular material, such as whole cells and cell debris).

Also, prior to the first chromatography step, a conditioning solution may be added to the cell broth or the antibody containing solution in order to ensure optimum conditions in terms of pH and conductivity for this first step.

In a particular embodiment the method according to the invention involves that the combined chromatography with AEX and MiMo is performed as a single unit operation.

In the context if the present invention with “antibody” and the plural “antibodies” is meant any protein which has the ability to specifically bind an antigen. In its natural form an antibody (or immunoglobulin) is a Y-shaped protein on the surface of B cells that is secreted into the blood or lymph in response to an antigenic stimulus, such as a bacterium, virus, parasite, or transplanted organ, and that neutralizes the antigen by binding specifically to it. The term antibody as used herein also comprises an antigen binding part of a natural or artificial antibody. The term antibody also comprises a non-natural (hence artificial) protein which has the ability to specifically bind to an antigen based on similar interaction mechanisms as a natural antibody, and therefore also comprises a chimeric antibody consisting e.g. of an antigen-binding part derived from one species (e.g. a mouse) and a non-antigen-binding part derived from another species (e.g. man).

With “mixed-mode chromatography (MiMo)” we mean that type of chromatography which makes use of materials in which more than one interaction takes place for the adsorption and/or desorption of proteins. These interactions may be of the following types: anionic, cationic, hydrophobic, affinity, π-π, thiophilic, size exclusion. Well known examples of mixed mode materials are hydroxyapatite (metal affinity, anionic and cationic interactions), Capto™ adhere (anionic and hydrophobic interactions) and MEP HyperCel™ (cationic and hydrophobic interactions).

With “serial, in-line AEX and MiMo” we mean that AEX and MiMo are serially connected in such a way that the outflow of the AEX device is fed into the MiMo device, without intermediate storage.

With “flow-through fraction” is meant here at least part of the loaded antibody-containing fraction which leaves the chromatographic column at substantially the same velocity as the elution fluid. This fraction is substantially not retained on the column during elution. Hence the conditions are chosen such that not the antibodies but the impurities are bound to the respective chromatographic materials.

It has been found that for large scale production purposes the method according to the present invention (with flow-through mode) provides a much faster separation than the prior disclosed method with binding and elution of the desired antibodies.

According to the present invention, the separation mixture containing the antibody is conditioned in-line. To this end the separation mixture is supplemented with an adequate amount of a suitable conditioning solution in order to alter its composition and/or properties, such as the pH and/or the conductivity and/or the presence and amounts of specific ionic components for optimum performance in the second chromatography step according to the present invention.

In none of the prior art documents cited above, in-line conditioning in between two chromatographic steps was applied nor suggested, and surprisingly it was found that very good separation results can be achieved with in-line conditioning of the fluid (separation mixture) before entering into the second chromatography step according to the invention.

Accordingly, the present invention relates to a method for the purification of antibodies from a protein mixture produced in a bioreactor, at least comprising the steps of intermediate purification and polishing, wherein the intermediate purification and polishing steps comprise serial in-line anion exchange chromatography (AEX), yielding as a flow-through fraction a separation mixture, followed by mixed-mode chromatography (MiMo) yielding as a flow through fraction a purified antibody preparation, and wherein the purified antibody preparation is subjected to at least one further purification step, wherein the separation mixture prior to mixed mode chromatography is supplemented with an adequate amount of a suitable adjusting solution in order to adjust the pH and/or conductivity and/or concentration or type of specific ionic components for removal of impurities from the antibodies in the mixed-mode chromatography step.

The terms “conditioning solution” and “adjusting solution” are used interchangeably and mean here the solution which is added to the separation mixture prior to feeding the separation mixture to the second (MiMo) chromatography step according to the invention.

With “an adequate amount of a suitable adjusting solution” is meant here any acidic, neutral or alkaline solution optionally containing one or more salts or any other additives that when mixed with the separation mixture will cause adsorption of the majority of relevant impurities to the MiMo material, but it will not promote substantial binding of the product. For each purification process the optimum pH, the preferred type of salt system and the optimum amounts in the adjusting solution have to be established.

Preferably, the pH of the mentioned solution will be the same as that of the separation mixture containing the antibody and the optimal conductivity value will be the result of the addition of an adequate amount of one or more salts or of dilution of the salt(s) present in the separation mixture. The anion of the salt may preferably be selected from the group consisting of phosphate, sulfate, acetate, chloride, bromide, nitrate, chlorate, iodide and thiocyanate ions. The cation of the salt may preferably be selected from the group consisting of ammonium, rubidium, potassium, sodium, lithium, magnesium, calcium and barium ions. Preferred salts are ammonium sulfate, sodium sulfate, potassium sulfate, ammonium phosphate, sodium phosphate, potassium phosphate, potassium chloride and sodium chloride. Other additives that may be used are ethanol, ethylene glycol, propylene glycol, polyethylene glycol or any other compound known in the art that serve to optimized the MiMo chromatography step.

The acidic components for an acidic adjusting solution may be chosen from compounds such as citric acid (or its mono or di basic sodium or potassium salts), phosphoric acid (or its mono or di basic sodium or potassium salts), acetic acid, hydrochloric acid, sulfuric acid.

The alkaline components for an alkaline adjusting solution may be chosen from compounds such as sodium or potassium hydroxide, (or its mono or di basic sodium or potassium salts), tris(hydroxymethyl)aminomethane, but any other alkaline component known in the art may be used to this end.

Preferably, the adjusting solution that is required will be supplemented in a small amount to have minimum dilution of the product.

Preferably, supplementing the separation mixture in this case with an adequate amount of an adequate adjusting solution is part of the single unit operation e.g. by in-line mixing of mentioned adjusting solution in the process stream (e.g. in a mixing chamber) prior to the MiMo chromatography step.

AEX chromatography according to the invention may take place in an AEX unit which may be embodied by a classical packed bed column containing a resin, a column containing monolith material, a radial column containing suitable chromatographic medium, an adsorption membrane unit, or any other anion exchange chromatography device known in the art with the appropriate medium and ligands to function as an anion exchanger. In the AEX column the chromatographic material may be present as particulate support material to which strong or weak cationic ligands are attached. The membrane-type anion exchanger consists of a support material in the form of one or more sheets to which strong or weak cationic ligands are attached. The support material may be composed of organic material or inorganic material or a mixture of organic and inorganic material. Suitable organic materials are agarose based media and methacrylate. Suitable inorganic materials are silica, ceramics and metals. A membrane-form anion exchanger may be composed of hydrophilic polyethersulfone containing AEX ligands. Suitable strong AEX ligands are based e.g. on quaternary amine groups. Suitable weak AEX ligands are based on e.g. primary, secondary or tertiary amine groups or any other suitable ligand known in the art.

MiMo chromatography according to the invention may take place in an MiMo unit which may be embodied by a classical column containing a resin, a column based on monolith material, a radial column containing suitable chromatographic medium, an adsorption membrane unit, or any other mix mode chromatography device known in the art with the appropriate ligands to function as a mixed mode material. In the MiMo column the chromatographic material may be present as particulate support material to which MiMo ligands are attached. The membrane-like chromatographic device consists of a support material in the form of one or more sheets to which MiMo ligands are attached. The support material may be composed of organic material or inorganic material or a mixture of organic and inorganic material. Suitable organic support materials are composed of e.g. hydrophilic carbohydrates (such as cross-linked agarose, cellulose or dextran) or synthetic copolymer materials (such as poly(alkylaspartamide), copolymers of 2-hydroxyethyl methacrylate and ethylene dimethacrylate, or acylated polyamine). Suitable inorganic support materials are e.g. silica, ceramics and metals. A membrane-form MiMo may be composed of hydrophilic polyethersulfone containing MiMo ligands. Suitable examples of MiMo ligands are hydroxyapatite, fluorapatite, 4-mercapto ethyl pyridine, hexylamino, phenylpropylamino, 2-mercapto-5-benzamidazole sulfonic acid, N-benzyl-N-methyl ethanolamine, or any other ligand known in the art with multimodal functionality.

Antibodies which can be purified according to the method of the present invention are antibodies which have an isoelectric pH of 6.0 or higher, preferably 7.0 or higher, more preferably 7.5 or higher. These antibodies can be immunoglobulins of the G, the A, or the M class. The antibodies can be human, or non-human (such as rodent) or chimeric (e.g. “humanized”) antibodies, or can be subunits of the abovementioned immunoglobulins, or can be hybrid proteins consisting of an immunoglobulin part and a part derived from or identical to another (non-immunoglobin) protein.

Surprisingly, the antibody material resulting from the combined AEX and MiMo chromatography generally will have a very high purity (referring to protein content) of at least 98%, preferably at least 99%, more preferably at least 99.9%, even more preferably at least 99.99%.

The AEX chromatography step according to the present invention preferably is carried out at neutral or slightly alkaline pH. It will remove the negatively charged impurities like DNA, host cell proteins, protein A (if present), viruses (if present), proteinacous medium components such as insulin and insulin like growth factor (if present).

During the MiMo chromatography step the major remaining large molecular impurities (mainly product aggregates) will be removed, using the property that, applying the right conditions of pH and conductivity, they bind to the chromatographic device while the product flows through.

Subsequently, the (highly) purified antibody preparation will, generally, have to be treated by ultrafiltration and diafiltration, in order to remove all residual low molecular weight impurities, to replace the buffer by the final formulation buffer and to adjust the desired final product concentration.

Furthermore, the purified antibody preparation will, generally, have to be treated also to assure complete removal of potentially present infectious agents, such as viruses and/or prions.

The present invention also relates to a single operational unit comprising both an anion exchange chromatography part (AEX) and a mixed mode chromatography part (MiMo), which are serially connected. This single operational unit further comprises an inlet at the upstream end of the first ion exchange chromatography part and an outlet at the downstream end of the second ion exchange chromatography part. This single operational unit also comprises a connection between the first ion exchange chromatography part and the second ion exchange chromatography part further comprising an inlet for supply of a conditioning solution to the separation mixture.

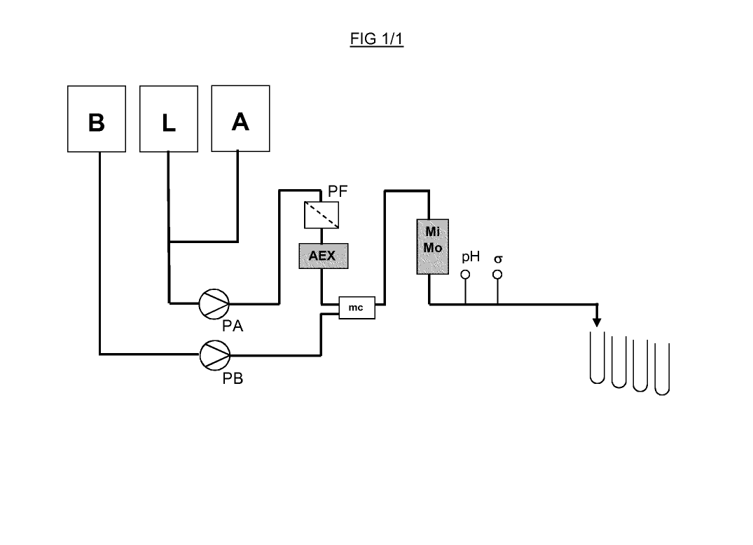

The liquid flow during the process according to the present invention can be established by any dual pump chromatographic system commercially available, e.g. an ÅKTA explorer (GE), a BIOPROCESS (GE) any dual pump HPLC system or any tailor made device complying with the diagram of FIG. 1. Most of these chromatographic devices are designed to operate a single chromatographic unit (i.e. column or membrane). With a simple adaptation, an extra connection can be made to place the first ion exchange unit after pump A and before the mixing chamber.

FIGS. 1 displays the basic configuration. Serial in-line connection of two chromatographic devices plus an optional pre-filter in the position as shown in FIG. 1, may occasionally lead to undesirable pressure buildup. Therefore, under some conditions extra technical adaptations (e.g. an extra pump after the AEX unit and a pressure reducing device before the AEX unit) may have to be included into this diagram.

### DESCRIPTION OF THE FIGURES

FIG. 1. A single operational unit comprising both an anion exchange chromatography part and a cation exchange chromatography part. Buffer A is a conditioning and washing buffer suitable for optimum operation of the AEX step. Buffer B contains an acidic solution and is mixed in a ratio to the load/buffer A required to obtain optimum conditions for operation of the MiMo step. The mixing ratio can be executed using a fixed volumetric mixing flow or can be automatically controlled by a feed back loop, based on e.g. the pH output. MC is an optional mixing chamber, which may contain any type of static mixer.
L=Load
PA=Pump A
PB=Pump B
AEX=anion exchange unit
MiMo=cation exchange unit
pH=pH sensor
σ=conductivity sensor
PF=optional pre-filter

### EXAMPLES

### Materials and Methods:

All experiments were carried out using an IgG produced by a CHO cell line. The cultivation was carried out in XD® mode, (see Genetic Engineering & Biotechnology news, Apr 1 2010, No. 7) using chemically defined medium.

Clarification and capture of the crude XD® harvest were carried out as single step using Rhobust® EBA technology with Protein A (see Innovations in Pharmaceutical Technology, March 2011). The product was eluted with 35 mM NaCl, 0.1 M Acetate; pH 3.0 elution buffer. The eluate contained 5 g/L IgG and was stored at 2-8° C.

With the material thus obtained, 6 experiments each were carried out: 1. to establish the conditions for preferential binding of aggregates in a MiMo chromatography using a hydroxyapatite resin (Experiment 1). 2. to run a MiMo chromatography using a hydroxyapatite resin in flow through mode with in-line mixing (Experiment 2). 3. to combine AEX and MiMo chromatography using a hydroxyapatite resin as one single unit operation (Example 1). 4. to establish optimum conditions in MiMo chromatography using an anionic-HIC resin in flow through mode (Experiment 3). 5. to run a MiMo chromatography using an anionic-HIC resin in flow through mode with in-line mixing (Experiment 4). 6. to combine AEX and MiMo chromatography using an anionic-HIC resin as one single unit operation (Example 2).

The optimum conditions for AEX chromatography in flow through mode, have been previously determined and were applied in the experiments of Example 1 and Example 2.

Protein (product) concentration was determined with UV/Vis spectroscopy by measuring absorbance at 280 nm (A²⁸⁰) and an extinction coefficient of 1.63.

Monomeric IgG and aggregate concentrations were determined by size exclusion chromatography (HP-SEC) according to standard procedures.

HCP was measured with the CHO HCP ELISA Assay, 3G (Cygnus Technologies)

### Experiment 1.

### Establishing the Conditions for Preferential Binding of Aggregates in a MiMo Chromatography Using a Hydroxyapatite Resin

For this experiment the pre-purified IgG was diluted with demineralized water to a conductivity of ≦5 mS/cm and was adjusted to pH 6.5 using a 2 M Tris pH 9.0. MiMo chromatography in bind-elute mode was carried out. A VL11 (Millipore) column filled with 4 cm bed length of HA Ultrogel® Hydroxyapatite Chromatography Sorbent (Pall, Life Sciences) was used on an \acute{Å}KTA explorer. The column was equilibrated and washed with a 10 mM sodium phosphate, pH 7.0 at a flow rate of 3 mL/min. The product was loaded at a flow rate of 2 mL/min. The initial load contained 2.6 g/L of IgG and an initial amount of aggregates of 2.2%. After loading, the product was eluted in a linear gradient from 0 to 100% with 10 mM sodium phosphate, pH 7.0 (buffer A) and 10 mM sodium phosphate, 1M NaCl, pH 7.0 (buffer B).

Fractions during the elution step were collected and analyzed for the presence of aggregates and protein (product) content as a function of conductivity.

TABLE 1
Aggregate elution in a hydroxyapatite resin with a sodium
phosphate/sodium chloride buffer at different conductivities

| | Conductivity | Aggregates | [IgG] |
|---|---|---|---|
| Fraction | mS/cm | % | g/L |
| A1 | 7.3 | 0 | 0.15 |
| A2 | 12.5 | 0 | 0.28 |
| A3 | 17.5 | 0 | 0.42 |
| A4 | 22.3 | 0 | 0.60 |
| A5 | 26.9 | 0.10 | 0.69 |
| A6 | 32.6 | 0.77 | 0.59 |
| A7 | 36.2 | 1.58 | 0.43 |
| A8 | 40.6 | 2.82 | 0.26 |

The analytical results on the samples (shown in Table 1) clearly indicated that up to a conductivity value of 26.9 mS/cm, the eluate does not contain or contains insignificant amounts of aggregates.

### Experiment 2.

Aggregate Removal in MiMo Chromatography Using a Hydroxyapatite Resin in Flow Through Mode with In-Line Mixing

For this experiment the pre-purified IgG was diluted with demineralized water to a conductivity of 2.4 mS/cm and was adjusted to pH 7.4 using a 2 M Tris pH 9.0 buffer. A VL11 (Millipore) column filled with 4 cm bed length of HA Ultrogel® Hydroxyapatite Chromatography Sorbent (Pall, Life Sciences) was used on an \acute{Å}KTA explorer. The column was equilibrated with demineralized water and 10 mM sodium phosphate, 0.8 M NaCl, pH 7.4 (buffer B). The demineralized water and buffer B were mixed in-line at fixed volume ratio of 30% buffer B, at a flow rate of 5 mL/min. After equilibration, the product was loaded. During loading the product flow was mixed in-line with buffer B in order to adjust the conductivity to a value of 25 mS/cm. The product flow and buffer B were mixed at fixed volume ratio of 30% of buffer B, at a 1 mL/min flow rate. The initial load contained 0.78 g/L of IgG and an initial amount of aggregates of 2.97%.

Fractions of the flow through were collected and analyzed for the presence of aggregates and protein (product) content.

TABLE 2
Aggregate clearance in a hydroxyapatite resin in flow through mode
with in-line mixing of a sodium phosphate/sodium chloride buffer

| | Aggregates | Total [IgG] |
|---|---|---|
| Fraction | % | g/L |
| A1 | 0.00 | 0.00 |
| A2 | 0.00 | 0.11 |
| A3 | 0.32 | 0.32 |
| A4 | 0.42 | 0.47 |
| A5 | 0.50 | 0.58 |
| A6 | 0.55 | 0.65 |
| A7 | 0.64 | 0.69 |
| A8 | 0.69 | 0.71 |
| A9 | 0.79 | 0.713 |
| A10 | 0.85 | 0.73 |
| A11 | 0.82 | 0.74 |
| A12 | 0.95 | 0.737 |

The analytical results of these samples (shown in Table 2) clearly indicated removal of aggregates to 5 1% using a hydroxyapatite resin in flow through mode with in-line mixing of the product containing load with a 10 mM sodium phosphate, 0.8 M NaCl, pH 7.4 at a fixed volume ratio of 30%.

### Example 1

Purification of IgG with AEX and MiMo Chromatography Using a Hydroxyapatite Resin as One Single Unit Operation

An AEX unit and a MiMo unit were serially coupled as depicted in the diagram of FIG. 1 using an \acute{Å}KTA explorer. For AEX, a Sartobind Q capsule (1 mL) was used and for the MiMo a VL11 (Millipore) column filled with 4 cm bed length of HA Ultrogel® Hydroxyapatite Chromatography Sorbent (Pall, Life Sciences) was used. For conditioning before product loading and prior to connecting the AEX unit, the MiMo unit was equilibrated with demineralized water (pumped with pump A) and 10 mM sodium phosphate, 0.8 M NaCl, pH 7.4 (buffer B). The demineralized water and buffer B were mixed in line at a fixed volume ratio of 30% of buffer B, at a flow rate of 5 mL/min. The AEX unit was flushed and equilibrated prior to connecting it to the system with 100 mL of 0.05 M Tris, pH 7.4 buffer. An experiment can be done in which equilibration of each unit is not done separately.

For this experiment the pre-purified IgG was diluted with demineralized water to a conductivity of 2.4 mS/cm, the pH was adjusted to pH 7.4 using a 2 M Tris pH 9.0 buffer and was filtered over 0.22 μm. The loading of the pre-purified IgG was started by pumping at a rate of 1 mL/min. Buffer B was pumped at the same flow rate at a 30% volume ratio. An amount of 240 mL containing 0.6 g/L of IgG was loaded. After completing the loading, the AEX unit was removed in order to start the wash. An experiment can be done in which the AEX unit does not need to be removed for the wash. The MiMo unit was washed with linear gradient from 0 to 30% of 10 mM sodium phosphate, pH 7.4 (buffer A) and buffer B and stripped with a 0.5 M sodium phosphate, 1.5 NaCl, pH 6.8 buffer. The load, the flow through and the wash were analyzed for the presence of aggregates, HCP content and protein (product) content. The load had an HCP concentration of 2179 ng/mg IgG. The flow through plus the wash fractions had a HCP concentration of 447 ng/mg IgG. The amount of aggregates in the load was 2.93% and was 0.76% in the flow through plus wash. The strip contained 54.97% of aggregates. The overall product recovery in the flow through plus wash was 88.2% and 90%in the flow through plus wash plus strip.

This experiment shows that a final purity of the antibody material of 99.2% is achieved by the use of serial in-line anion exchange chromatography followed by MiMo (hydroxyapatite) chromatography operating as one single unit operation when the separation mixture is supplemented in-line with an adequate amount of an adequate adjusting solution. The initial purity of the load was 97%

### Experiment 3.

### Establishing Optimum Conditions in MiMo Chromatography Using an Anionic-HIC Resin in Flow Through Mode

For this set of experiments the pre-purified IgG was diluted with demineralized water to a conductivity of 2.29 mS/cm, the pH was adjusted to pH 7.4 using a 2 M Tris pH 9.0 buffer. A VL11 (Millipore) column filled with 6.3 bed length Capto™ adhere (GE Healthcare) was used was used on an \acute{Å}KTA explorer. The column was equilibrated and washed with 25 mM sodium phosphate, pH 7.4, (buffer A) and 100 mM sodium phosphate, pH 7.4 (buffer B). Buffer A and buffer B were mixed in line at 0, 5, 15 and 25% volume ratio, at a flow rate of 5 mL/min as separate runs. After equilibration, the product was loaded. During loading the product flow was mixed in-line with buffer B. The product flow and buffer B were mixed in-line at a volume ratio of 0, 5, 15 and 25% of buffer B at a flow rate of 3 mL/min as separate runs. The initial load contained 1.09 g/L of IgG prior to dilution due to in-line mixing with buffer B and an initial amount of aggregates of 3.13%. The column was stripped with a 100 mM sodium phosphate, pH 3.0 buffer.

Fractions of the flow through at different ratios of buffer B were collected and analyzed for the presence of aggregates and protein (product) content.

TABLE 3
Aggregate clearance in an anionic-HIC MiMo resin in flow
through mode using a sodium phosphate buffer at different ratios

| Buffer B | Aggregates in the FT | Total [IgG] |
|---|---|---|
| % | % | mg/MI |
| 0 | 1.15 | 1.04 |
| 5 | 0.23 | 0.88 |
| 15 | 0.18 | 0.82 |
| 25 | 0.17 | 0.76 |

The analytical results of the samples (shown in Table 3) clearly indicated removal of aggregates to <1% in an anionic-HIC MiMo resin when the product containing load is mixed in-line with a phosphate salt adjusting buffer.

### Experiment 4.

Aggregate Removal in MiMo Chromatography Using an Anionic-HIC Resin in Flow Through Mode with In-Line Mixing

For this experiment the pre-purified IgG was diluted with demineralized water to a conductivity of 2.4 mS/cm and was adjusted to pH 7.4 using a 2 M Tris pH 9.0 buffer. A VL11 (Millipore) column filled with 6.3 bed length Capto™ adhere (GE Healthcare) was used on an \acute{Å}KTA explorer. The column was equilibrated and washed with 25 mM sodium phosphate, pH 7.4, (buffer A) and 100 mM sodium phosphate, pH 7.4 (buffer B). Buffer A and buffer B were mixed in-line at a fixed volume ratio 15% buffer B, at a flow rate of 5 mL/min. After equilibration, the product was loaded. During loading the product flow was mixed in-line with buffer B. The product flow and buffer B were mixed in-line at a fixed volume ratio of 15% of buffer B at a flow rate of 3 mL/min. The initial load contained 0.93 g/L of IgG and an initial amount of aggregates of 3.15%. The column was stripped with a 100 mM sodium phosphate, pH 3.0 buffer.

TABLE 4
Aggregate clearance in an anionic-HIC MiMo resin in flow
through mode with in-line mixing of a sodium phosphate buffer

| | Aggregates in FT | Total [IgG] |
|---|---|---|
| Fractions | % | mg/mL |
| A2 | 0.00 | 0.011 |
| A3 | 0.00 | 0.054 |
| A4 | 0.23 | 0.204 |
| A5 | 0.19 | 0.456 |
| A6 | 0.16 | 0.659 |
| B7 | 0.16 | 0.761 |
| B6 | 0.15 | 0.829 |
| B5 | 0.17 | 0.853 |
| B4 | 0.16 | 0.852 |
| B3 | 0.17 | 0.859 |
| B2 | 0.16 | 0.865 |
| B1 | 0.19 | 0.861 |
| C1 | 0.18 | 0.853 |
| C2 | 0.22 | 0.856 |
| C3 | 0.20 | 0.855 |

The analytical results of these samples (shown in Table 4) clearly indicated removal of aggregates to ≦1% in the flow through throughout the run in an anionic-HIC MiMo resin in flow through mode with in-line mixing of a 100 mM sodium phosphate, pH 7 at a fixed volume ratio of 30%. The aggregate percentage is the bulk of the flow through was 0.18%

### Example 2.

Purification of IgG with AEX and MiMo Chromatography Using an Anionic-HIC Resin as One Single Unit Operation

An AEX unit and a MiMo unit were serially coupled as depicted in the diagram of FIG. 1 using an \acute{Å}KTA explorer. For AEX, a Sartobind Q capsule (1 mL) was used and for the MiMo a VL11 (Millipore) column filled with 6.3 bed length Capto™ adhere (GE Healthcare) was used. For conditioning before product loading and prior to connecting the AEX unit, the MiMo unit was equilibrated with 25 mM sodium phosphate, pH 7.4, (buffer A) and and 100 mM sodium phosphate, pH 7.4 (buffer B). Buffer A and buffer B were mixed in-line at a fixed volume ratio of 15% buffer B, at a flow rate of 5 mL/min. The AEX unit was flushed and equilibrated prior to connecting it to the system with 100 mL of 0.05 M Tris, pH 7.4 buffer. An experiment can be done in which equilibration of each unit is not done separately.

For this experiment the pre-purified IgG was diluted with demineralized water to a conductivity of 2.29 mS/cm, the pH was adjusted to pH 7.4 using a 2 M Tris pH 9.0 buffer and was filtered over 0.22 μm. The loading of the pre-purified IgG was started by pumping at a rate of 3 mL/min. Buffer B was pumped at the same flow rate at a 15% volume ratio. An amount of 479 mL containing 0.91 g/L of IgG was loaded. After completing the loading, the AEX unit was removed and the flow was switched back to Buffer A and the line was primed, in order to start the wash. An experiment can be done in which the AEX unit does not need to be removed for the wash. After washing, the MiMo unit was stripped by adding a 100 mM sodium phosphate, pH 3.0 buffer via pump A and pump B was stopped. The load, the flow through and the wash were analyzed for the presence of aggregates, HCP content and protein (product) content. The load had an HCP concentration of 1711 ng/mg IgG. The flow through plus the wash fractions had a HCP concentration of 206 ng/mg IgG. The amount of aggregates in the load was 3.13% and was 0.18% in the flow through plus wash. The strip contained 14.23% of aggregates. The overall product recovery in the flow through plus wash was 82.9% and 99.9% in the flow through plus wash plus strip.

This experiment shows that a final purity of the antibody material of 99.72% is achieved by the use of serial in-line anion exchange chromatography followed by MiMo (anionic-HIC) chromatography operating as one single unit operation when the separation mixture is supplemented in-line with an adequate amount of an adequate adjusting solution. The initial purity of the load was 96.8%

### Abbreviations Used

A280 (Light) Absorption at 280 nm
AEX Anion Exchange
BHK cells Baby Hamster Kidney cells
CHO cells Chinese Hamster Ovary cells
EBA Expanded Bed Adsorption
HCP Host Cell Protein
HIC Hydrophobic Interaction Chromatography
HPLC High Pressure Liquid Chromatography
IgG Immunoglobulin G
MiMo Mixed Mode
TFF Tangential Flow Filtration
Tris tris(hydroxymethyl)methylamin

1. Method for the purification of antibodies from a protein mixture produced in a bioreactor, at least comprising the steps of intermediate purification and polishing, wherein the intermediate purification and polishing steps comprise serial in-line anion exchange chromatography (AEX) and mixed-mode chromatography (MiMo) both in flow-through mode, wherein the AEX step yields as a flow-through fraction a separation mixture containing antibodies, wherein the separation mixture is subjected to a step without intermediate storage, yielding as a flow through fraction a purified antibody preparation, and wherein the purified antibody preparation is subjected to at least one further purification step, wherein the separation mixture prior to the MiMo step is supplemented with an adequate amount of a suitable adjusting solution in order to adjust the pH and/or conductivity and/or concentration or type of specific ionic components for removal of impurities from the antibodies in the step.

2. Method according to claim 1 wherein anion exchange chromatography and mixed mode chromatography take place in two separate devices which are serially connected.

3. Method according to claim 1 wherein the serial in-line AEX and MiMo are performed as a single unit operation.

4. Method according to claim 1 wherein the separation mixture prior to MiMo is supplemented with an adequate amount salt or a combination of salts.

5. Method according to claim 4 wherein the separation mixture prior to MiMo is supplemented with an adequate amount of ammonium sulfate, sodium sulfate, potassium sulfate, ammonium phosphate, sodium phosphate, potassium phosphate, potassium chloride and sodium chloride.

6. Method according to claim 1 wherein the separation mixture prior to MiMo chromatography is supplemented with an adequate amount of an acidic solution.

7. Method according to claim 6 wherein the separation mixture prior to MiMo chromatography is supplemented with an adequate amount of a solution containing citric acid (or its monobasic or dibasic sodium or potassium salts), phosphoric acid (or its monobasic or dibasic sodium or potassium salts), acetic acid, hydrochloric acid or sulfuric acid.

8. Method according to claim 1 wherein the separation mixture prior to mixed mode chromatography is supplemented with an adequate amount of an alkaline solution.

9. Method according to claim 8 wherein the separation mixture prior to MiMo chromatography is supplemented with an adequate amount of a solution containing sodium or potassium hydroxide, (or its mono or di basic sodium or potassium salts) or tris(hydroxymethyl)aminomethane

10. A single operational unit which can be used in a method according to claim 1 comprising both an anion exchange chromatography part and a mixed mode chromatography part, which are serially connected, wherein the outlet of the anion exchange chromatography part is connected to the inlet of the mixed mode chromatography part, wherein the unit comprises an inlet at the upstream end of the anion exchange chromatography part and an outlet at the downstream end of the mixed mode chromatography part and wherein the unit also comprises an inlet between the anion exchange chromatography part and the mixed mode chromatography part.
